(19) United States
(12) Patent Application Publication
(10) Pub. No.: US 2011/0027124 A1
(43) Pub. Date: Feb. 3, 2011

(54) Electric fragrance diffuser

(76) Inventors: Eric M. Albee, Lamberville, NJ (US); Sandra J. Keeley, Bensalem, PA (US); Kathryn Ludwig, New York, NY (US); Heidi Valentino, New York, NY (US)

Correspondence Address: WARD & OLIVO, SUITE 300, 382 SPRINGFIELD AVENUE, SUMMIT, NJ, 07901, US

(21) Appl. No.: 12/462,115

(22) Filed: Jul. 29, 2009

(51) Int. Cl. A61L 9/00

(52) U.S. Cl. 422/5; 422/124

(57) ABSTRACT

The electric fragrance diffuser including a housing having a plurality of outlet and inlet vents, a chamber for holding a fragrance medium, and a fan for drawing air from the inlet vents and creating an updraft airflow which facilitates the emission of the fragrance from the fragrance medium. The electric fragrance diffuser also includes a cowling structure for increasing and directing airflow in an upward direction. The cowling structure further interacts with inlet vents in the housing for accelerating the airflow in an upward direction and thereby accelerating the emission of the fragrance from the fragrance medium, wherein the fragrance is released from the apparatus via the outlet vents. The electric fragrance diffuser further comprises a storage compartment for storing at least one additional fragrance medium.

### FIELD OF THE INVENTION

The present invention generally relates to an electric fragrance diffuser and in particular to a new and useful electric fragrance diffuser comprising a fan and a cowling structure for effectively releasing fragrance into the air.

### BACKGROUND OF THE INVENTION

Air diffusers, also known as air fresheners, have existed for some time. Generally, an air freshener emits an aroma into a room or enclosed area. The aroma may create a mood, invoke a psychological response and/or mask unpleasant odors. In some instances, aromas are used for therapeutic purposes. For example, aromatherapy is a form of alternative medicine that employs the aromas of volatile essential oils for the purpose of affecting a person's mood or health, mentally, emotionally, and spiritually. Essential oils are concentrated aromatic oils obtained from plants, flowers, and herbs. Different oils have different therapeutic effects when the aromas are inhaled. The most common way of using essential oils in aromatherapy is by dispersing the aroma via various diffusing systems. As stated above, such systems may also be used to mask unpleasant odors.

Previous methods of diffusing essential oils used candle diffusers. Candle diffusers typically include a candleholder in the lower part of the diffuser and a reservoir above the candle to contain the oil. A candle, typically a tea-light, is placed in the candleholder to heat the oil above, causing the oil to evaporate into the air. However, candle diffusers are dangerous to use because of the risk of fire, and therefore should not be left unattended.

Electric diffusers that are inexpensive to manufacture and which diffuse fragrances into the air without any risk of fire have become available. These electric diffusers receive power from either a household electrical outlet, or a battery. They generally comprise a receptacle for containing the fragrance oil and an electric heater that heats the oil causing it to evaporate into the air. These diffusers are capable of dispersing fragrance continuously into the air, while providing the benefit of remaining unattended, without the risk of starting a fire.

A common problem with heat-activated diffusers (i.e., candle diffusers, electric diffusers, etc.) is that the smell of the produced fragrance is altered as essential oils are burned when heated. Burned oils may incur health hazards, since some burned components in the essential oils may be toxic. The burning of the oils may also alter the beneficial effects of the oils. Thus, cold air diffusers are generally preferred.

A nebulizer is one form of a cold air diffuser. It consists of a base containing a motor, whereby the base is constructed from blown-glass. The nebulizer breaks the essential oil into separate molecules before dispersing them into the air. Although this method is said to have high therapeutic value, nebulizers are expensive and are difficult to maintain. The blown-glass piece is easily breakable and expensive to replace. In addition, some nebulizers are incompatible with oils that have heavy particle sizes.

An alternative method of dispersing fragrance oils without heat is by using fan-based electric diffusers. Typically, these systems comprise a housing, an air inlet and an air outlet, a battery-operated fan, and a container having liquid therein. In operation, the fan forces air past a fragrance contained within the diffuser, thereby creating airflow between the air inlet and the air outlet. The airflow causes the evaporated liquid in the container to travel through the diffuser, whereby the fragrance is finally emitted into the air from the diffuser via the air outlet. The liquid is contained in any suitable container. One known container includes a bottle with a wick. In such a design, the partially submerged portion of the wick absorbs the liquid, some of which is drawn up by capillary or wicking action to the exposed, non-submerged portion of the wick where the liquid evaporates into the surrounding air. Another known container includes a cartridge containing permeable material. The liquid contained in such cartridge is impregnated in the permeable material and drawn up by capillary action to its surface where the liquid evaporates into the air.

A known disadvantage of the fan-based systems is that they are incapable of dispersing high concentrations of the fragrance into the central area of a room. One effort to fix this problem was to include a louver structure, disposed upstream of the fan. The louver structure is generally angled upwardly and away from the fan for directing the airstream created by the fan upwardly and away from the upper portion of the fragrance container. Although this structure assists in directing the airflow of the fragrance, it does not affect the force of the airflow.

In addition, the aforementioned systems use containers that are prone to spillovers. Known air freshener systems generally contain a breakable fragrance bottle or cartridge that is exposed to the user at all times. Breakable fragrance bottles exposed to the user are prone to falling and subsequently shattering. Similarly, the cartridges may be disengaged, whereby the liquid may spill outside the air freshener. The liquid fragrance may drip on the floor or furniture and create a harmful, dangerous and/or unsanitary environment. In addition, these fragrance systems implement special containers that are made to fit the fragrance system and are only available through one supplier. As such, only a limited variety of fragrances is available, and if the supplier discontinues the item, the user is left with an unusable device.

In light of the shortcomings described above concerning fan-based fragrance diffusers, there is a clear need for a more efficient method of directing the airflow within the diffuser in order to maximize the efficiency of the diffuser as well as maximize fragrance dispersion. There is also a clear need for a fragrance diffuser that allows a user to utilize any essential oil via an absorption pad or fragrance medium system.

### SUMMARY OF THE INVENTION

It is an object of the present invention to provide an electric fragrance diffuser that is cost efficient.

In addition, it is an object of the present invention to provide an electric fragrance diffuser having an aesthetically pleasing design.

It is also an object of the present invention to provide an electric fragrance diffuser that releases aroma without the use of heat or a flame.

It is also an object of the present invention to provide an electric fragrance diffuser that utilizes a fan to diffuse fragrance into the air.

It is still another object of the present invention to provide a cowling that creates an airflow updraft suction effect, which directs and maximizes airflow through the fragrance medium in order to more efficiently release aroma.

It is yet another object of the present invention to provide a fragrance diffuser that is not limited to certain fragrance containers or specific scents.

It is also an object of the present invention to energize a fan in order to continuously release a high concentration of a fragrance into the air.

It is also an object of the present invention to utilize a drawer and a fragrance medium to prevent any liquid fragrance from escaping the housing.

It is yet another object of the present invention to store additional unused fragrance mediums within the housing of the fragrance diffuser.

It is also object of the present invention to provide a fragrance diffuser that allows a user to easily change or replace a fragrance medium.

The present invention preferably includes a housing comprising air outlet apertures or vents, air inlet apertures or vents, and a chamber for holding a fragrance medium. The chamber is preferably removable from the housing to allow for ease of replacing the fragrance medium. The apparatus further comprises a fan for creating an upstream airflow and facilitating the emission of the fragrance from the fragrance medium, the airflow entering the inlet vents and exiting the outlet vents. The housing further contains a cowling structure that increases airflow and directs airflow in an upward direction. Thereby, the cowling structure directs and accelerates the emission of a fragrance from the fragrance medium.

The chamber for holding the fragrance medium optimally contains at least one aperture for facilitating the release of fragrance from the fragrance medium and a tab for coupling the chamber to the apparatus. The fragrance medium is preferably made from a permeable material capable of absorbing fragrance, such as fragrance oil. However, fragrance medium may also consist of a fragrance absorption pad, a saturated felt pad, a scented solid, a membrane gel tray, potpourri, or ceramic scented beads.

The apparatus also includes a storage compartment removably attached to the housing. The storage compartment of the present invention selectively couples to the housing and preferably contains at least one unused fragrance medium.

Additionally, the apparatus comprises an electrical circuit receiving current from a power source to energize the fan, wherein the power source is preferably a battery. The apparatus further comprises a cavity for receiving the battery. The apparatus further comprises a switch to activate the fan and a light source to indicate to the user that the apparatus is active.

Other objects, features, and characteristics of the present invention, as well as the methods of operation and functions of the related elements of the structure, and the combination of parts and economies of manufacture, will become more apparent upon consideration of the following detailed description with reference to the accompanying drawings, all of which form a part of this specification.

### SUMMARY OF THE DRAWINGS

A further understanding of the present invention can be obtained by reference to a preferred embodiment, along with some alternative embodiments, set forth in the illustrations of the accompanying drawings. Although the illustrated embodiments are merely exemplary of systems for carrying out the present invention, the organization and method of operation of the invention in general, together with further objectives and advantages thereof, may be more easily understood by reference to the drawings and the following description. The drawings are not intended to limit the scope of this invention, which is set forth with particularity in the claims as appended or as subsequently amended, but merely to clarify and exemplify the invention.

For a more complete understanding of the present invention, reference is now made to the following drawings.

FIG. 1 illustrates a front perspective view of an electric fragrance diffuser in accordance with one embodiment of the present invention.

FIG. 2 illustrates an exploded view of the electric fragrance diffuser shown in FIG. 1 in accordance with one embodiment of the present invention.

FIG. 3 illustrates a top perspective view of the middle section of the electric fragrance diffuser shown in FIG. 1 in accordance with one embodiment of the present invention.

FIG. 4 illustrates a bottom plan view of the middle section of the electric fragrance diffuser shown in FIG. 1 in accordance with one embodiment of the present invention.

FIG. 5 illustrates a top perspective view of the drawer of the electric fragrance diffuser shown in FIG. 1 in accordance with one embodiment of the present invention.

FIG. 6 illustrates a bottom perspective view of the bottom section of the electric fragrance diffuser shown in FIG. 1 in accordance with one embodiment of the present invention.

FIG. 7 illustrates a top perspective view of the storage compartment shown in FIG. 1 in accordance with one embodiment of the present invention.

FIG. 8 illustrates a cross-sectional view of the electric fragrance diffuser shown in FIG. 1 in accordance with one embodiment of the present invention.

### DETAILED DESCRIPTION OF THE INVENTION

A detailed illustrative embodiment of the present invention is disclosed herein. However, techniques, systems and operating structures in accordance with the present invention may be embodied in a wide variety of forms and modes, some of which may be quite different from those in the disclosed embodiment. Consequently, the specific functional details disclosed herein are merely representative, yet in that regard, they are deemed to afford the best embodiment for purposes of disclosure and to provide a basis for the claims herein which define the scope of the present invention.

Moreover, well known methods and procedures for both carrying out the objectives of the present invention and illustrating the preferred embodiment are incorporated herein but have not been described in detail as not to unnecessarily obscure novel aspects of the present invention.

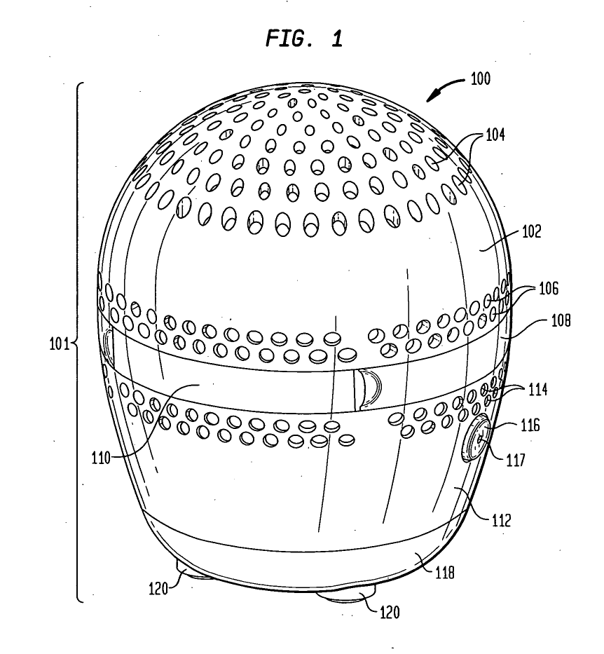

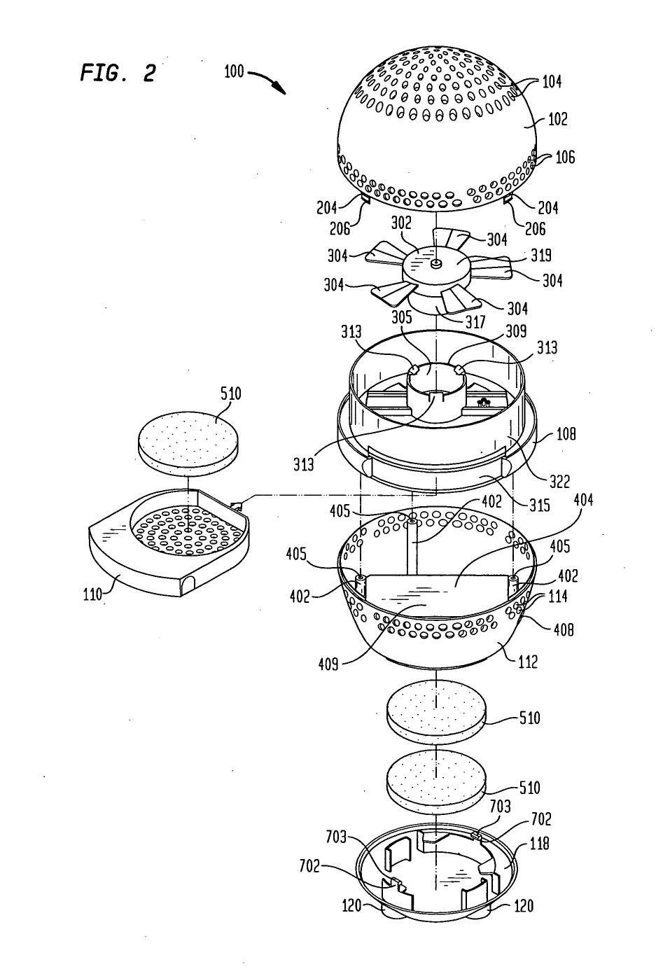

Referring to FIGS. 1-2, the electric fragrance diffuser of the present invention is generally designated 100, and comprises a multi-part hollow housing 101. Preferably, housing 101 comprises a top section 102, a middle section or center band 108, a bottom section 112, and a storage compartment 118. Electric fragrance diffuser 100, housing 101, top section 102, middle section 108, bottom section 112, and storage compartment 118 are preferably constructed from any sturdy plastic as commonly known in the art. Preferably, top section 102, middle section 108, bottom section 112, and storage compartment 118 are each formed from injection molded plastic such that each comprise a single molded piece. However, each component may be made of any appropriate material without departing from the spirit of the present invention. Moreover, as shown in FIG. 1, each of these components are preferably coupled together to form a continuous, single piece design, for example an egg shape design. While a particular shape and appearance of electric fragrance diffuser 100 and its components have been illustrated in FIGS. 1-2, it should be appreciated that any shape or design may be implemented without limiting the scope of the present invention.

As shown in FIGS. 1-2, top section 102 is semi-spherically shaped and preferably contains a plurality of apertures or outlet vents 104. Outlet vents 104 are preferably provided on the upper portion of top section 102. Outlet vents 104 primarily allow for the release of an aroma or fragrance from diffuser 100. Additionally, outlet vents 104 are substantially circular in shape. However, outlet vents 104 may be any appropriate shape or size and may be in any appropriate location. Furthermore, any appropriate number of outlet vents 104 may be implemented in the design without limiting the scope of the present invention. Top section 102 also contains a plurality of apertures or air inlet vents 106, which are substantially circular in shape and are disposed right above the lower edge of top section 102. Specifically, air inlet vents 106 encircle the perimeter of the top section 102 along it lower edge. Air inlet vents 106 preferably allow air to be pulled into diffuser 100, which assists in increasing the airflow velocity and directing the airflow within fragrance diffuser 100. Accordingly, the air that enters diffuser 100 via air inlet vents 106 assists in facilitating the release of an aroma from diffuser 100. This process will be described in further detail below. Air inlet vents 106 may be any appropriate shape or size, and may be in any appropriate location. Additionally, any appropriate number of air inlet vents 106 may be implemented in the design without limiting the scope of the present invention.

As shown in FIG. 2, top section 102 further comprises a plurality of coupling members 204 protruding in a downward direction from the bottom edge of top section 102. Each coupling member 204 is preferably molded to the internal surface of top section 102, whereby each coupling member 204 extends from internal surface of top section 102, thus forming one molded piece of plastic. It should be appreciated that coupling members 204 may be attached to top section 102 by any appropriate means commonly known in the art without limiting the scope of the present invention. Furthermore, each coupling member 204 preferably comprises a flange 206 to engage and snap-fit into each respective aperture or sockets 308 (FIG. 4) disposed within middle section 108 in order to couple top section 102 to middle section 108.

As shown in FIGS. 2-4, middle section 108 is cylindrical in shape and preferably contains a drawer 110, fan 302 and a cowling 322. Moreover, as shown in FIGS. 1-2, the upper portion of middle section 108 is preferably coupled to the bottom portion of top section 102. Specifically, middle section 108 preferably comprises an external ring 307. External ring 307 preferably contains a plurality of sockets 308 to be used to couple top section 102 to middle section 108. During the coupling of middle section 108 to top section 102, top section 102 is placed over middle section 108 and each coupling member 204 is aligned with each respective socket 308. Once each coupling member 204 is aligned with its respective socket 308, each coupling member 204 engages and slides through each respective socket 308 until each flange 206 securely latches to the underside of external ring 307, thus securely coupling top section 102 to middle section 108. Accordingly, when top section 102 is coupled to middle section 108, fan 302 and cowling 322 are disposed within top section 102 of diffuser 100.

Referencing FIG. 2, fan 302 is preferably a standard fan commonly used in the art, whereby fan 302 preferably comprises a hub 319, a plurality of fan blades 304 and a motor 317 disposed thereunder. Hub 319 is circular in shape, wherein fan blades 304 are circumferentially disposed around thereof. The proximate end of each fan blade 304 is preferably molded to hub 319, whereby the distal end of each fan blade extends outwardly from hub 319. In order to facilitate the rapid rotation of fan blades 304, hub 319 is preferably placed on top of motor 317 where hub 319 is rotatably coupled to a center axel (not shown) of motor 317 as commonly known in the art. Fan 302 and fan blades 304 are preferably constructed from a single piece molded plastic. However, either component may be constructed from any appropriate material without limiting the scope of the present invention.

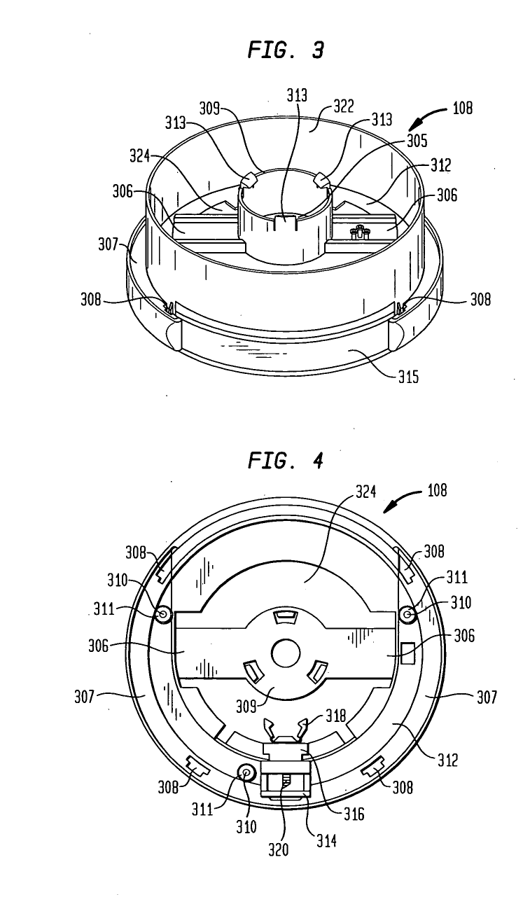

Referencing FIG. 3, middle section 108 preferably comprises an internal ring 312, which is preferably coupled to the internal surface thereof. A fan support 306, which contains a fan housing 309, is preferably coupled to internal ring 312. Preferably, fan housing 309 comprises cavity 305 and a plurality flanges 313 in order to receive and retain fan motor 317 so as to securely maintain fan 302 within housing 101. The plurality of flanges 313 are preferably equidistantly spaced apart from each other along the top edge of fan housing 309 and are coupled to fan housing 309 through the process of molding plastic components together as commonly known in the art. Preferably, motor 317 slides into cavity 305 until each flange 313 engages the top surface of motor 317 located just within the underside of hub 319, wherein hub 319 is positioned there above. Once each flange 313 has engaged the top surface of motor 317, motor 317 becomes securely retained within cavity 305.

As further shown in FIGS. 2-3, middle section 108 comprises cowling 322. Cowling 322 is preferably constructed from plastic and is coupled on its lower edge to external ring 307 of middle section 108. Moreover, cowling 322 is preferably coupled to external ring 307 through a method of molding plastic as commonly known in the art. As shown in FIG. 8, cowling 322 is preferably completely encloses fan 302, whereby cowling 322 is located in close proximity to the tips of fan blades 304. In addition, fan blades 304 are substantially enclosed by cowling 322, but can extend slightly above the top edge of cowling 322. This maximizes fan's 302 efficiency as cowling 322 increases the velocity of the airflow. As is further shown in FIG. 8, cowling 322 is preferably located in close proximity with the inner side wall of upper section 102 so that external ring 307 peripherally extends from cowling 322 towards the inner wall of top section 102. Thereby, cowling 322 and external ring 307 substantially enclose the air inlet vents 106 for preventing downward airflow, but leaving an escape space 801 thereabove. The functionality of cowling 322 will be described below in further detail.

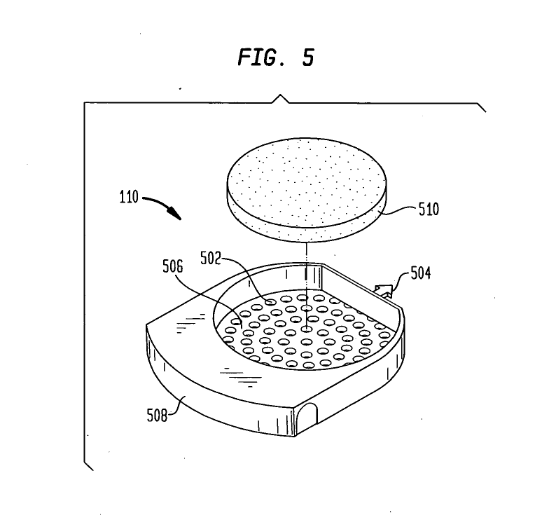

With further reference to FIGS. 2-5, middle section 108 further comprises an opening 315 and a cavity 324. Preferably, opening 315 is large enough to slidably receive drawer 110. Once drawer 110 enters opening 315, cavity 324 preferably receives and retains drawer 110. Specifically, middle section 108 preferably comprises a cavity 314 disposed within internal ring 312. Cavity 314 houses coupling member 316. Coupling member 316 comprises at least two prongs 318 and a spring 320. As shown in FIG. 5, drawer 110 comprises a tab 504. Preferably coupling member 316 is used to receive a tab 504 attached to drawer 110 in order to couple drawer 110 to diffuser 100. Once a user slides drawer 110 into cavity 324, the user may then push front surface 508 of drawer 110 so that tab 504 fits within prongs 318 of coupling member 316. The user can continue to push front surface 508 so that tab 504 activates the spring-loaded mechanism of coupling member 316, whereby tab 504 becomes locked into place and drawer 110 subsequently remains disposed within cavity 324 of middle section 108. To release tab 504 from coupling member 316, the user can push front surface 508 thereby releasing the spring-loaded mechanism within coupling member 316. Once tab 504 is released from coupling member 316, the drawer is pushed outward by the spring-loaded mechanism and the user may remove drawer 110 from cavity 324. While a specific method and mechanism is described for selectively coupling and disposing drawer 110 within diffuser 100, it should be appreciated that any method and mechanism commonly known in the art may be use to couple and dispose drawer 110 within diffuser 100 without limiting the scope of the present invention.

As further shown in FIG. 5, drawer 110 comprises a plurality of apertures 502 and a cavity 506. As previously described, drawer 110 is generally disposed within cavity 324. In a preferred embodiment of the present invention, a user places a fragrance medium 510 in cavity 506. Fragrance medium 510 is preferably made from a permeable material capable of absorbing fragrance, such as fragrance oil. Once drawer 110 is disposed within middle section 108, apertures 502 allow air to enter the bottom of cavity 506 to facilitate the release of a fragrance into the air. Fragrance medium 510 may also consist of a fragrance absorption pad, a saturated felt pad, a solid scented piece, a membrane gel tray, potpourri, ceramic scented beads, or any appropriate medium commonly known in the art. Fragrance medium 510 may be made of cotton, polyester, plastic or any appropriate material without departing from the spirit of the present invention. The fragrance medium may be impregnated with any fragrance oil commonly available on the market for use with fragrance oil burners or diffusers. The fragrance, oils may be purchased from any supplier and are available in a wide variety of fragrances. Moreover, since this configuration allows the fragrance oil to be stored hidden within the diffuser, the electric fragrance diffuser of the present invention eliminates spills and potentially hazardous conditions created by other fragrance diffusers.

Referencing FIG. 4, internal ring 312 of middle section 108 preferably comprises a plurality of coupling members 311 containing cavities 310. Coupling members 311 are used to be engaged with coupling members 402 of bottom section 112 (FIG. 2) in order to couple middle section 108 to bottom section 112. As shown in FIGS. 1-2, coupling members 402 of bottom section 112 are generally cylindrical in shape and protrude from the internal bottom surface of bottom section 112. Preferably, each coupling member 402 contains a hole 405, which serves to receive and hold a screw. Preferably, middle section 108 is coupled to bottom section 112 by first aligning each coupling member 402 of bottom section 112 with each respective coupling member 311 of middle section 108. Once aligned, each coupling member 402 of bottom section 112 enters and engages each cavity 310 of each coupling member 311 of middle section 108. Each coupling member 402 continues to slide through each respective cavity 310 until it engages and rests against an internal flange. Once each coupling member 402 has been fully inserted into each respective cavity 310, a screw is inserted into the top side of each cavity 310 and subsequently tightened into each aperture 405 of coupling member 402, thereby securely coupling middle section 108 to bottom section 112. While a specific method for coupling middle section 108 to bottom section 112 has been described above, any appropriate method may be implemented for doing the same without limiting the scope of the invention.

As shown in FIGS. 1-2, bottom section 112 is cylindrical in shape, and tapers downwardly. Bottom section 112 preferably comprises a plurality of apertures or air inlet vents 114, which are substantially circular in shape and are disposed right below the upper edge of bottom section 112. Additionally, air inlet vents 114 allow air to be pulled into diffuser 100 and create airflow throughout fragrance diffuser 100. Moreover, the air that enters air inlet vents 114 is pulled past a fragrance medium 510 resting within drawer 110 in order to draw the fragrance therefrom. Accordingly, the air that enters diffuser 100 via air inlet vents 114 assists in facilitating the release of an aroma from diffuser 100. This process will be described in further detail below. Air inlet vents 114 may be any appropriate shape or size, and may be in any appropriate location. Furthermore, any appropriate number of air inlet vents 114 may be implemented in the design without limiting the scope of the present invention.

Bottom section 112 also preferably contains an actuation switch 116. Preferably, actuation switch 116 is a push button design, whereby electric diffuser 100 is activated when a user depresses switch 116. It should be appreciated that switch 116 may be any type of switch commonly known in the art (i.e., an on/off push button switch, an on/off sliding switch, a light sensing switch, etc.) without limiting the scope of the present invention. Additionally, actuation switch 116 may include a light source 117, whereby light source 117 may be any type of light source commonly known in the art, such as a light emitting diode. Accordingly, once a user depresses switch 116, light source 117 becomes active and remains active for as long as switch 116 is closed. Thus, light source 117 preferably serves to notify the user that diffuser 100 is currently active. It should be appreciated that light source 117 may be located anywhere within diffuser 100 without departing from the spirit of the present invention. It should also be appreciated that light source 117 may provide additional functions, for example, it may function as a battery life indicator.

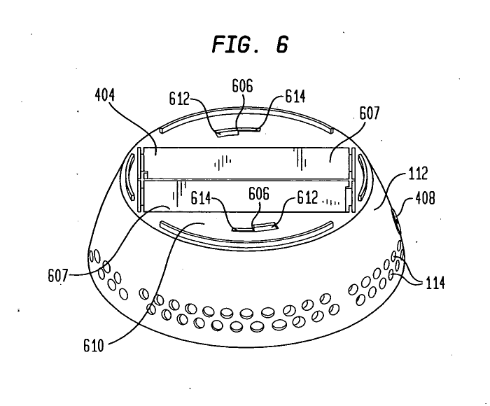

Referring now to FIG. 6, illustrated is an underside view of bottom section 112, which comprises an aperture 408 for receiving the actuation switch 116 (FIG. 1) and an external surface 610 with battery cavity 404. In the current design of diffuser 100, battery cavity 404 contains slots 607 used to store two size AA batteries. It should be appreciated that any size slot(s) can be provided to store any size or type of battery without limiting the scope of the present invention. The battery is preferably used to energize an electric circuit (not shown), whereby the electric circuit is coupled to fan 302, switch 116, and light source 117. However, other power sources can be also be used without departing from the scope of the present invention. For example, bottom section 112 may include an additional aperture (not shown) for receiving a power connector that connects the circuit to a power cord that plugs into an electrical outlet or a USB cord that plugs into a computer. In any configuration, once switch 116 is actuated the circuit closes, thus transferring electricity from the power source (e.g., the battery) to both fan 302 and light source 117 thereby activating diffuser 100. While not shown, the electric circuit may be coupled to the bottom surface 409 (FIG. 2) of bottom section 112. However, the electric circuit may be placed in any appropriate location without limiting the scope of the present invention.

As shown in FIG. 6, the underside of bottom section 112 further comprises retaining apertures 606. Retaining apertures 606 are constructed to be cutouts in external surface 610 of bottom section 112. Preferably, the retaining apertures 606 each contain two distinct sections varying in width. Specifically, each retaining aperture 606 preferably contains a wide section 612 and a narrow section 614, whereby the sections are designed to facilitate the coupling and removal of storage compartment 118 to and from bottom section 112 (FIG. 2).

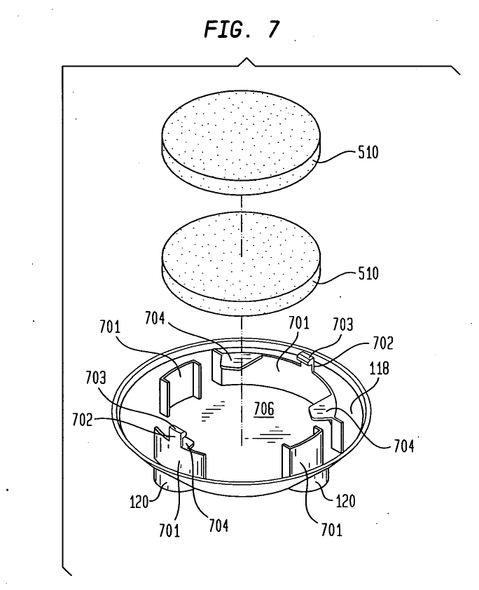

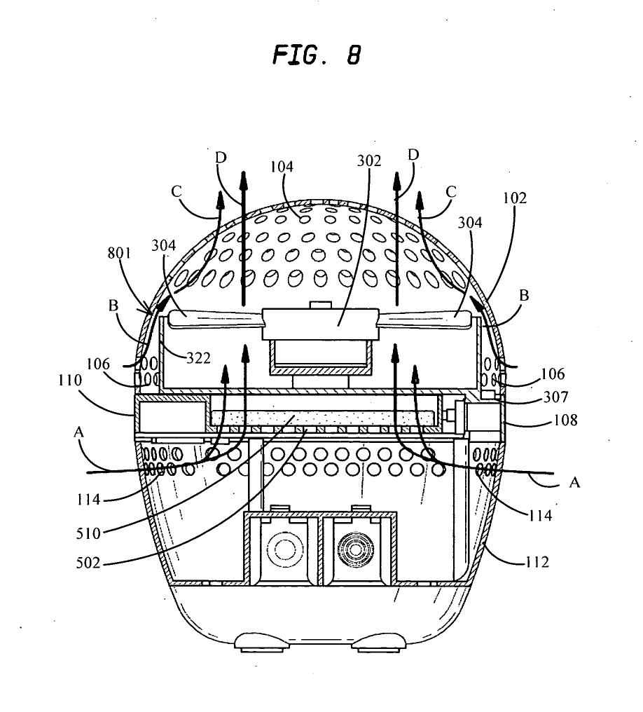

As shown in FIG. 7, storage compartment 118 is cylindrical in shape and comprises a plurality of retaining members 701 and at least two coupling members 702. Coupling members 702 serve to couple storage compartment 118 to bottom section 112 (FIG. 6). Preferably, each coupling member contains a flange 703. Referring to FIGS. 6-7, in order to couple storage compartment 118 to bottom section 112, a user may first align each coupling member 702 with wide section 612 of each respective retaining aperture 606. The user may then slide each coupling member 702 through wide section 612 of each respective retaining aperture 606. Once each coupling member 702 has traveled completely through its respective retaining aperture 606, the user can then rotate storage compartment 118 in a clockwise direction, thereby sliding each coupling member 702 into narrow section 614 of each respective retaining aperture 606. Once each coupling member 702 enters narrow section 614, flange 703 engages the interior surface (not shown) of bottom section 112, thereby securely coupling storage compartment 118 to bottom section 112.

To release storage compartment 118 from bottom section 112, a user can rotate storage compartment 118 in a counter clockwise direction until each coupling member 702 enters the wide section 612 of each respective retaining aperture 606, whereby the user then is able to pull storage compartment 118 away from bottom section 112. The method for coupling and releasing storage compartment 118 to and from bottom section 112 as described above is commonly known in the art and any method may be used without departing from the spirit of the present invention.

As shown in FIG. 7, retaining members 701 extend from the upper surface of storage compartment 118 and are spaced far enough apart so as to create a large enough cavity 706 to hold unused or additional fragrance mediums 510. Retaining flanges 704 are coupled to the top portion of retaining members 701. As shown in FIG. 7, a retaining flanges 704 are not provided on each retaining member 701. However, it should be appreciated that each retaining member 701 may contain a retaining flange 704 without limiting the scope of the present invention. Once a fragrance membrane 510 has been placed into cavity 706, retaining flanges 704 serve to securely maintain fragrance membrane 510 within cavity 706.

It should be appreciated that any number of retaining structures 701, coupling members 702, and retaining flanges 704 may be implemented in the design of diffuser 100 without limiting the scope of the present invention.

Further referring to FIG. 7, storage compartment 118 comprises at least two feet 120 located on the bottom surface of storage compartment 118. Preferably, storage compartment 118 contains four feet 120. Preferably, each foot 120 contains a disk located on its tip in order to provide friction between diffuser 100 and a surface on which it is placed so that diffuser 100 may securely rest on that surface. The friction created between the disk and the surface diffuser 100 is resting on assists in stabilizing diffuser 100 and allows diffuser 100 to stand upright on the surface without slipping or falling over. The disk may be any appropriate material commonly known in the art without limiting the scope of the present invention, for example fabric, felt, plastic, composite material, etc. Moreover, it should be appreciated that any number of feet 120 may be implemented in the design without limiting the scope of the present invention.

While each section of housing 101 has been outlined in detail above, it should be appreciated that the sections that form housing 101 may be coupled together in any order and by any appropriate means without limiting the scope of the present invention. In addition, while the storage compartment 118 and drawer 110 is readily removable by the user, preferably, top section 102, middle section 108, and bottom section 112 are securely engages to each other and are not readily removable. This will ensure that the user does not temper with the inner pieces of the diffuser 100, such as the fan and the circuit.

Referencing FIGS. 1-7, in operation, a user may first remove storage compartment 118 from bottom section 112. Once storage compartment 118 is removed, the user may place batteries (not shown) into battery cavity 404 and remove a fragrance medium from storage compartment 118. The user may then reattach storage compartment 118 to bottom section 112. The user may subsequently remove drawer 110 from cavity 324 by gently pushing front surface 508, thereby releasing drawer 108 from coupling member 316. The user may then pull drawer 110 out far enough from diffuser 100 so that the user is able to place fragrance medium 510 within cavity 506 of drawer 110. Once fragrance medium 510 is placed within cavity 506, the user may provide a fragrance to the fragrance medium 510 by preferably distributing drops of fragrance oil evenly onto the fragrance medium 510. Moreover, it should be appreciated that any type of fragrance oil commonly used in the art may be implemented with the present invention without limiting its scope. Once the fragrance oil has been applied to the fragrance medium 510, the user may return drawer 110 to its original position within diffuser 100 by placing it into opening 315 and gently pushing it inside cavity 324 until it is securely locked into position.

Once diffuser 100 has been properly prepared for activation as described above, the user may then place diffuser 100 on a stable surface. To actuate diffuser 100, the user may preferably press switch 116. Upon pressing switch 116, light source 117 becomes active and fragrance diffuser 100 begins to release an aroma into the air. During operation, fragrance diffuser 100 is energized by receiving electricity from the batteries (not shown). Electrical power is drawn from the batteries (not shown) to fan 302 and light source 117. The batteries are preferably connected to fan 302 and light source 117 by means of electrical conducting wire (not shown) and basic electrical components (not shown) commonly used in the art.

Referring now to FIG. 8, once fan 302 is energized fan blades 304 rotate at a high rate of speed, whereby fan 302 and cowling 322, in combination, draw air into diffuser 100 via air inlet vents 106 and 114. Specifically, air current A enters air inlet vents 114 and is pulled up through apertures 502 in drawer 110 due to the updraft created by fan 302 and cowling 322. Specifically, the pitch of the fan blades 304 and the rotation of the fan 302 pulls the air upwards and the encasing of the fan within cowling 322 increases the velocity of the airflow, thus maximizing the efficiency of the fan 302. Air current A travels through and captures the fragrance from fragrance medium 510 disposed within drawer 110. Preferably, cowling 322 creates an updraft vacuum or suction effect to pull fragrance volatiles from fragrance medium 510, whereby the fragrance is subsequently released into the air. This forces air current B to be drawn into diffuser 100 via air inlet vents 106 and through space 801 between cowling 322 and the side wall of upper section 102. As shown in FIG. 8, air current B is drawn upwards into the upper portion of top section 102, wherein air current C subsequently results. Preferably, air current C facilitates the release of fragrance from diffuser 100 into the air by increasing airflow velocity and directing the airflow upward towards outlet vents 104. As fan blades 304 rapidly rotate, air current D is generated from a combination of the air current created by fan 302, air current A, air current B, and air current C, wherein air current D is directed upward towards outlet vents 104. Preferably, air current D and air current C flow through outlet vents 104, subsequently releasing the fragrance captured in air current A from diffuser 100. Accordingly, air current D and air current C in combination preferably facilitate the release of the fragrance from diffuser 100.

The method of creating this updraft suction effect includes the combination of forced airflow from fan 302, which is encased in cowling 322 to direct and maximize airflow and a balanced level of air inlet venting to allow effective, though not excessively open airflow. Moreover, cowling 322 captures the air currents generated by fan 302 and focuses this airflow in a chosen direction (i.e., in an upward direction), thereby generating air force, focused airflow and suction in order to create a focused airflow system. The placement of cowling 322 (i.e., its close proximity to fan 302) assists in maximizing its efficiency in generating and directing airflow within diffuser 100. Cowling 322 preferably functions to control the air currents generated by the rotating fan and directs the air currents C and D upward. By keeping cowling 322 in close proximity to the tips of fan blades 304, the air currents generated are prevented from dissipating and are directed upward based on the pitch of fan blades 304. Through the placement of fan 302 and the utilization of cowling 322 along with the placement of air inlet vents 106 and 114, diffuser 100 acts as a suction airflow system that pulls air past a fragrance medium and releases that fragrance into the air.

While fan blade pitch alone can create airflow, the use of cowling 322 in the present invention collects the air to focus and intensify the airflow. The combination of the focused airflow generated from cowling 322, with the proper amount of air inlet venting, along with the placement of fragrance medium 510 within diffuser 100, creates a suction effect within diffuser 100. Furthermore, the created suction effect pulls fragrance volatiles from the fragrance medium and releases these volatiles into the air. In order to effectively direct and intensify the airflow within diffuser 100 the distance between cowling 322 and the tips of fan blades 304 should be minimal. However, it should be appreciated that the space between cowling 322 and the tips of fan blades 304 may be any appropriate distance without limiting the scope of the present invention. Preferably, the focused airflow system, which focuses airflow within diffuser 100 and creates a forced air suction effect as described above, is created through the use of cowling 322 in combination with the general air intake dynamics of the present invention.

It should be appreciated, that while fragrance medium 510 is located below fan 302 and cowling 322, a person of ordinary skill in the art can position fragrance medium 510 in other location with respect to fan 302 and cowling 322 as long as it is located within the updraft airflow. For example, fragrance medium 510 may be located above fan 302 and cowling 322.

Electric fragrance diffuser 100 is also cost efficient, since the fragrance medium only requires a few drops of oil or fragrance. Conversely, other diffusers consume a significant amount of oil or fragrance, which can become an expensive interest for, those who wish to be surrounded by aromas all the time.

While the present invention has been described with reference to the preferred embodiment and alternative embodiments, which have been set forth in considerable detail for the purposes of making a complete disclosure of the invention, such embodiments are merely exemplary and are not intended to be limiting or represent an exhaustive enumeration of all aspects of the invention. The scope of the invention, therefore, shall be defined solely by the following claims. Further, it will be apparent to those of skill in the art that numerous changes may be made in such details without departing from the spirit and the principles of the invention. It should be appreciated that the present invention is capable of being embodied in other forms without departing from its essential characteristics.

1. A fragrance diffusing apparatus, comprising:
a hollow housing including a top wall having outlet vents, a side wall having a plurality of first inlet vents and a plurality of second inlet vents, and a bottom wall, said plurality of first inlet vents being disposed above said plurality of second inlet vents forming a space therebetween;
a spring-loaded, fully removable chamber disposed in said space adapted for retaining a fragrance medium;
a generally horizontal cowling structure disposed above said chamber including a cylindrical vertical wall disposed in close proximity to said plurality of first inlet vents;
a fan disposed within said cowling structure comprising a plurality of fan blades substantially encased in said cylindrical vertical wall, said fan creates an airflow; and,
a storage compartment removably attached to said housing for storing at least one additional fragrance medium.

2. The apparatus according to claim 1, wherein said fan and said cowling structure draw air into said housing via said plurality of second inlet vents to facilitate the emission of a fragrance from said fragrance medium.

3. The apparatus according to claim 2, wherein said fan and said cowling structure further draw air into said housing via said plurality of first inlet vents for increasing airflow velocity and facilitating the emission of said fragrance from said housing.

4. The apparatus according to claim 2, wherein said fragrance is released from said housing via said outlet vents.

5. The apparatus according to claim 1, wherein said housing comprises separate sections, including a top section, a middle section, and a bottom section.

6. The apparatus according to claim 5, wherein said outlet vents and said plurality of first inlet vents are disposed in said top section, wherein said chamber is disposed in said middle section, and wherein said plurality of second inlet vents are disposed in said bottom section.

7. The apparatus according to claim 1, further comprising a fan housing for retaining said fan within said cowling structure.

8. The apparatus according to claim 1, wherein said cowling structure further comprises an external ring extending peripherally from a lower edge of said cylindrical vertical wall to an inner surface of said side wall of said housing.

9. The apparatus according to claim 8, wherein said cylindrical vertical wall and said external ring substantially enclose said plurality of first inlet vents for preventing downward airflow.

10. The apparatus according to claim 1, wherein said chamber is removable from said housing for replacing said fragrance medium.

11. The apparatus according to claim 1, wherein said chamber comprises a drawer.

12. The apparatus according to claim 1, wherein said chamber includes at least one aperture for allowing airflow to pass through said fragrance medium.

13. The apparatus according to claim 1, wherein said housing further includes a coupling member for coupling said chamber to said housing.

14. The apparatus according to claim 13, wherein said chamber is inserted inside said housing, and further wherein said chamber remains locked in place via said coupling member.

15-16. (canceled)

17. The apparatus according to claim 1, wherein said fragrance medium is a permeable material capable of absorbing fragrance.

18. The apparatus according to claim 1, wherein said fragrance medium is a fragrance absorption pad, a saturated felt pad, a scented solid, a membrane gel tray, potpourri, or a plurality of ceramic scented beads.

19. A fragrance diffusing apparatus including a housing, comprising:
a first section, said first section including at least one first aperture and at least one second aperture; said at least one first aperture allows air to enter said housing and said at least one second aperture allows air to exit said housing;
a second section coupled to said first section, said second section including a fan, an airflow directing and accelerating member, and a cavity to receive a spring-loaded, fully removable chamber for holding a fragrance medium;
a third section coupled to said second section, said third section including at least one third aperture, said at least one third aperture allows air to enter said housing; and,
a fourth section coupled to said third section, said fourth section including a storage compartment removably attached to said fourth section for storing at least one additional fragrance medium.

20. A fragrance diffusing apparatus, comprising:
a hollow housing including at least one outlet vent, at least one first inlet vent, and at least one second inlet vent;
a spring-loaded, fully removable chamber disposed in said housing for retaining a fragrance medium;
a fan disposed in said housing for creating an airflow and facilitating the emission of a fragrance from said fragrance medium, said airflow entering said at least one first inlet vent and exiting said at least one outlet vent;
an airflow directing means disposed above said chamber for increasing and directing said airflow in an upward direction;
an air accelerating means for further increasing the velocity of said airflow via air entering through said at least one second inlet vent; and
a storage compartment removably attached to said housing for storing at least one additional fragrance medium.

21. The apparatus according to claim 20, wherein said chamber is removable from said housing for replacing said fragrance medium.

22. The apparatus according to claim 20, wherein said chamber comprises a drawer.

23. The apparatus according to claim 20, wherein said chamber includes at least one aperture for allowing said airflow to pass through said fragrance medium.

24-25. (canceled)

26. The apparatus according to claim 20, wherein said fragrance medium is a permeable material capable of absorbing said fragrance.

27. The apparatus according to claim 20, wherein said fragrance medium is fragrance absorption pad, a saturated felt pad, a scented solid, a membrane gel tray, potpourri, or a plurality of ceramic scented beads.

28. A fragrance diffusing apparatus, comprising:
a hollow housing including at least one outlet vent and at least one inlet vent;
a spring-loaded, fully removable chamber disposed in said housing adapted for retaining a fragrance medium comprising a permeable material capable of absorbing a fragrance;
a fan for creating an airflow and facilitating the emission of said fragrance from said fragrance medium, said airflow entering said at least one inlet vent and exiting said at least one outlet vent; and,
a storage compartment removably attached to said housing for containing at least one additional fragrance medium.

29. The apparatus according to claim 28, wherein said chamber is removable from said housing for replacing said fragrance medium.

30. The apparatus according to claim 28, wherein said chamber comprises a drawer.

31. The apparatus according to claim 28, wherein said chamber includes at least one aperture for allowing said airflow to pass through said fragrance medium.

32. A fragrance diffusing apparatus, comprising:
a hollow housing including a top wall having at least one outlet vent, a side wall having a plurality of first inlet vents and a plurality of second inlet vents, and a bottom wall, said plurality of first inlet vents being disposed above said plurality of second inlet vents;
a spring-loaded, fully removable chamber disposed in said housing adapted for retaining a fragrance medium;
a generally horizontal cowling structure disposed above said second inlet vents including a cylindrical vertical wall disposed in close proximity to said plurality of first inlet vents;
a fan disposed within said cowling structure comprising a plurality of fan blades substantially encased in said cylindrical vertical wall, said fan creates an airflow; and,
a storage compartment removably attached to said housing for storing at least one additional fragrance medium.

33. The apparatus according to claim 32, wherein said cowling structure further comprises an external ring extending outwardly and peripherally from said cylindrical vertical wall for separating said plurality of first inlet vents from said plurality of second inlet vents.

34. A method for diffusing a fragrance, comprising the steps of:
providing a hollow housing including at least one outlet vent, at least one first inlet vent, and at least one second inlet vent;
retaining a fragrance medium within a spring-loaded, fully removable chamber disposed in said housing;
creating an airflow for facilitating the emission of a fragrance from said fragrance medium via a fan, said airflow entering said at least one first inlet vent and exiting said at least one outlet vent;
increasing and directing said airflow in an upward direction via a cowling structure disposed above said chamber;
increasing the velocity of said airflow via air entering through said at least one second inlet vent;
providing a storage compartment removably attached to said housing for storing at least one additional fragrance medium.

35. The method according to claim 34, further comprising removing said chamber from said housing to replace said fragrance medium.

36. The method according to claim 34, wherein said airflow passes through said fragrance medium via at least one aperture contained in said chamber.

37-38. (canceled)

39. The method according to claim 34, further comprising absorbing said fragrance via said fragrance medium.
